(19) United States
(12) Patent Application Publication
(10) Pub. No.: US 2004/0013737 A1
(43) Pub. Date: Jan. 22, 2004

(54) Taste masked oral composition of telithromycin

(76) Inventors: Philippe Becourt, Massy (FR); Nicoletta Cioloca, Sevran (FR); Luigi Boltri, Agrate Brianza (IT); Stefano De Luigi Bruschi, Milano (IT); Luigi Giovanni Mapelli, Milano (IT); Leonardo Rabaglia, Parme (IT); Detlev Schwabe, Garches (FR)

Correspondence Address: Finnegan, Henderson, Farabow,, Garrett & Dunner, L.L.P., 1300 I Street, N.W., Washington, DC, 20005-3315, US

(21) Appl. No.: 10/198,115

(22) Filed: Jul. 19, 2002

(51) Int. Cl. A61K 31/7048; A61K 31/7008; A61K 9/14; A61K 9/16; A61K 9/50

(52) U.S. Cl. 424/490; 514/29; 514/62

(57) ABSTRACT

The invention relates to an oral compositions comprising telithromycin that have taste masking properties, a process for the preparation thereof, and methods of using the compsition.

This application claims the benefit of patent ______ application No. ______ filed Jul. 19, 2001 by inventors that are likely to include Luigi Boltri, Stefano De Luigi Bruschi, Giovanni Mapelli Luigi, and Leonardo Rabaglia and ______ relating to oral compositions comprising spherical agglomerates of an active ingredient, ethylcellulose, at least one acrylic polymer and at least one anti-agglomeration agent (e.g. talc) and a process for the preparation thereof. The priority application is herein incorporated by reference.

The invention relates to an oral compositions comprising telithromycin that have taste masking properties, a process for the preparation thereof, and methods of using the compsition.

Telithromycin is an already known antibacterial active described in EP 680,967, the contents of which are herein incorporated by reference. Unfortunately, it has an unpleasant taste and therefore it cannot be formulated in a simple oral composition, mainly for paediatric use. So far it has not be found how to overcome the problem of preparing a composition that could be acceptable.

International application WO 01/14393, the contents of which are herein incorporated by reference, describes spherical agglomerates of telithromycin that are obtained by direct transformation of crystals into spherical forms. These agglomerates have a size of between 30 and 400 μm and preferentially 80 to 150 μm. The described agglomerates are used to prepare micro-capsules for the preparation of oral suspensions, using a coacervation process. Unfortunately, when using the general methods of micro-encapsulation and coacervation, it is impossible to obtain a composition with sufficient properties of taste masking.

It has now be found that using specific components it is possible to prepare a composition of telithromycin whose taste is acceptably masked from an organoleptic point of view and has, after reconstitution, good stability properties and acceptable bioavailability.

An embodiment of the inventive composition comprises microcapsules comprising two coating layers: a first coating comprising ethylcellulose and a second coating comprising a layer of an acrylic polymer. Thus, in one embodiment, this invention provides improved pharmaceutical formulations where the unpleasant taste is masked while giving an immediate delivery release in the stomach. In a further embodiment, the composition can advantageously be administered in the form of a suspension for oral administration.

Thus, for example, in one embodiment, the inventive composition comprises spherical agglomerates of telithromycin, ethylcellulose, at least one acrylic polymer and at least one anti-agglomeration agent. In another embodiment, the composition comprises spherical agglomerates of telithromycin, ethylcellulose, at least one acrylic polymer, at least one anti-agglomeration agent, D(-) N-methylglucamine, and aluminium and magnesium silicate. The compositions of the invention may further comprise at least one optional agents chosen from suspending agents, aromatization agents, sweetening agents and/or antimicrobial preservation agents.

In one embodiment, the compositions of the invention are in the form of microcapsules comprising two coating layers. As used herein, microcapsules comprising two coating layers includes microcapsules comprising additional coating layers. In one embodiment, the first coating of said two coating layers is an ethylcellulose coating and a second coating of said two coating layers is an acrylic polymer coating.

Another embodiment of the invention is a process for preparing a composition of telithromycin, comprising forming telithromycin into spherical agglomerates, microencapsulating the spherical agglomerates with ethylcellulose, and fluid bed coating the microcapsules with at least one acrylic polymer. The microcapsules may, for example, be made by coacervation in cyclohexane. In one embodiment, the process may further comprises adding D(-) N-methylglucamine, aluminium silicate and magnesium silicate. The process may also comprise adding at least one component chosen from suspending agents, aromatization agents, sweetening agents and antimicrobial preservation agents.

Another embodiment of the invention is a process for preparing a composition of telithromycin, comprising forming telithromycin into spherical agglomerates, microencapsulating the spherical agglomerates with ethylcellulose, fluid bed coating the microcapsules with at least one acrylic polymer and adding D(-) N-methylglucamine, aluminium silicate, magnesium silicate, xanthane gum, maltitol, sodium saccharinate and flavouring agent.

In another embodiment, the invention provides a method of treating bacterial based infections comprising administering to a patient in need thereof an effective amount of an inventive composition. The invention also provides, for example, for a method for oral administration of telithromycin comprising orally administering an inventive composition to a patient.

Another embodiment of the invention is a method for taste masking telithromycin comprising combining in the form of microcapsules comprising two coating layers spherical agglomerates of telithromycin, ethylcellulose, at least one acrylic polymer, and at least one anti-agglomeration agent.

It is to be understood that both the foregoing general description and the following detailed description are exemplary and explanatory only and are not restrictive of the invention, as claimed.

In one embodiment, the acrylic polymer can be chosen, for example, from Eudragit® E. The anti-agglomeration agent may, for example, be talc. An example of a suspending agentsis xanthan gum. The aromatization agents may, for example, be chosen from sugars and for instance maltitol. The antimicrobial preservation agent may, for example, be chosen from parabens, and the sweetening agent may be chosen for instance from sodium saccharinate.

Example amounts of components for use in the practice of the invention include (in % by weight of the total weight of the composition):

spherical agglomerates of telithromycin present in an amount ranging from 40 to 65%,

ethylcellulose present in an amount ranging from 8 to 18%,

acrylic polymer present in an amount ranging from 18 to 35%,

talc present in an amount ranging from 4.5 to 10%,

D(-) N-methylglucamine present in an amount ranging from 0.8 to 1.8%,

aluminium and magnesium silicate present in an amount ranging from 4 to 9%,

Example amounts of optional addatives or further components for use in the practive of the invention include (in % by weight of the total weight of the composition):

xanthan gum present in an amount ranging from 0.3 to 0.7%,

maltitol present in an amount ranging from 40 to 90%,

sodium saccharinate present in an amount ranging from 0.6 to 1.4%, and

flavouring agent present in an amount ranging from 0.6 to 1.4%.

In one embodiment, the composition of the invention comprises (in % by weight of the total weight of the composition), spherical agglomerates of telithromycin present in an amount ranging from 40 to 65%, ethylcellulose present in an amount ranging from 8 to 18%, acrylic polymer present in an amount ranging from 18 to 35%, talc present in an amount ranging from 4.5 to 10%, D(-) N-methylglucamine present in an amount ranging from 0.8 to 1.8%, aluminium and magnesium silicate present in an amount ranging from 4 to 9% and optionally the above mentioned additives.

In another embodiment, the composition of the invention comprises (in % by weight of the total weight of the composition) spherical agglomerates of telithromycin present in an amount ranging from 45 to 63%; ethylcellulose present in an amount ranging from 9 to 15%; acrylic polymer present in an amount ranging from 18 to 33%; talc present in an amount ranging from 5 to 8%, D(-) N-methylglucamine present in an amount ranging from 0.8 to 1.8%, aluminium and magnesium silicate present in an amount ranging from 4 to 9% and optionally the above mentioned additives.

Also, according to an embodiment of the invention, the ratio of acrylic polymer/anti-agglomeration agent may be chosen from around 2 to 4, for example 4.

In one embodiment, a suitable mean size of the telithromycin agglomerates used as starting material can range from 30 and 500 μm. For example, the mean size can range from 60 and 250 μm (as determined by light scattering analysis).

According to the invention, the composition is prepared from telithromycin in the form of spherical agglomerates (as described in WO 01/14393) using two steps of encapsulation.

first step: microencapsulation with ethylcellulose,

second step: fluid bed coating of the microcapsules with the acrylic polymer.

In another embodiment, where the composition comprises D(-) N-methylglucamine, aluminium and magnesium silicate and optionally suspending agents, aromatization agents, sweetening agents and/or antimicrobial preservation agents, these optional additives are added in a third step of the process, for example, after the two encapsulation steps. In yet another embodiment, where the composition comprises D(-) N-methylglucamine, aluminium and magnesium silicate and optionally xanthane gum, maltitol, sodium saccharinate and flavouring agent, these additives can also be added in a third step of the process, preferably, but not necessarily, after the two encapsulation steps.

The two steps of encapsulation can be conducted as follows:

microencapsulation in ethylcellulose can be made by coacervation according to the method described in U.S. Pat. No. 6,139,865 and EP 38585 (both incorporated here by reference), with ethylcellulose in cyclohexane and polyethylene wax, followed by separation of the microcapsules from the liquid phase and drying on a fluid bed.

the encapsulation in an acrylic polymer can be made using fluid bed coating technics, in the presence of an anti-agglomeration agent. The process is carried out in a hydro alcoholic medium (for example in hydro-ethanolic medium).

Examples of components include: the polyethylene wax can be Epolen®, the acrylic polymer can be chosen among Eudragit E (such as Eudragit® E100), the anti-agglomeration agent can be talc and, in one example, a micronized talc or talc of less than 75 μm.

The amount of the primary coating with ethylcellulose is preferably, but not necessarily, an amount that will achieve optimal particule size distribution and will provide microcapsules that are suitable to be coated using a fluid bed technic. Residual solvents should comply with the regulatory acceptance.

In one embodiment, an amount ranging from 15 and 30% of coating level is suitable, such as, for example, a coating level ranging from 18 and 25% and such as 18%. In one embodiment, the microcapsules prepared with the primary coating comprise 50 to 95% by weight of telithromycin agglomerates and 5 to 50% by weight of ethylcellulose. After the first coating the mean particule size is, for example, less than 250 μm for more than 90% of the resulting microcapsules.

The secondary coating, made of acrylic polymer in the presence of an anti-agglomeration agent is made, for example, in an amount that allows at least one for the following: a narrow particule size distribution, satisfactory taste-masking and fast dissolution rate for the preparation of the suspension. In one embodiment, an amount between 20 and 45% of coating level is suitable, such as for example, a coating level between 25 and 40%. After the second coating the mean particule size is, for example, less than 300 μm for more than 90% of the resulting microcapsules.

It is possible to prepare from the microcapsules, suspensions comprising from 5 to 250 mg/ml and more preferably 100 mg/ml. In one embodiment, the prepared microcapsules have the capability of forming a sufficiently stable suspension, and have a fast dissolution rate equal or superior to 80% over 2 to 10 minutes and in some aspects of the invention a dissolution rate of 90% within 2 nm.

In a further embodiment, it has been shown that compositions of the invention provide a satisfactory taste masking up to five days after suspension reconstitution.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Unless otherwise indicated, all numbers expressing quantities, reaction conditions, and so forth used herein are to be understood as being modified in all instances by the term “about.” Accordingly, unless indicated to the contrary, the numerical parameters set forth in the specification herein and in the attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The following examples illustrate the invention.

### EXAMPLE 1

The first step of coating was carried out using a 5 liter reactor with a pneumatic stirring, introducing spherical agglomerates of telithromycin with ethylcellulose in cyclohexane and Epolene®, with agitation at a temperature of 80° C., according to the coacervation method described in U.S. Pat. No. 6,139,865.

The resulting microcapsules were sieved

After carrying out the first step, the second coating was performed over 75 mn, with using a Glatt GPCG1 fluid bed equipped with Wurster insert (4″), bottom plate <<A >> type, filter <<T165P>> type, 1 mm spraying nozzle and adding to 456 g of the first coated microcapsules, Eudragit®) E100 10%, talc <75 μm 5.0%, ethanol 51%, purified water 34%. The coating layer was applied at a spraying rate of 2.5 to 5.5 g/mn. The temperature of the air was 45 to 52° C. and the air speed 1.0 to 1.5 m/s. The atomisation pressure was 1.8 bars. The temperature of the product during the coating was between 30 and 33° C. The coating was performed with an amount of membrane of 25%. After drying on the fluid bed and sieving the corresponding 2 coatings microcapsules are obtained. The resulting composition comprises (by weight) telithromycin agglomerates 58.5%, ethylcellulose 6.5%, Eudragit®) E 23.3%, talc 11.7%. The coated samples were easily dispersible in aqueous medium without any agglomeration or segregation phenomena.

### EXAMPLE 2

After carrying out a first step according to the example 1, the second coating was performed over 92 mn, with using a Glatt GPCG1 fluid bed equipped with Wurster insert (4″), bottom plate <<A>> type, filter <<T165P>> type, 1 mm spraying nozzle and adding to 456 g of the first coated microcapsules, Eudragit®) E100 10%, talc <75 μm 5.0%, ethanol 51%, purified water 34%. The coating layer was applied at a spraying rate of 2.0 to 5.0 g/mn. The temperature of the air was 54 to 55° C. and the air speed 1.0 to 1.5 m/s. The atomisation pressure was 1.8 bars. The temperature of the product during the coating was between 30 and 33° C. The coating was performed with an amount of membrane of 35%. After drying on the fluid bed and sieving the corresponding 2 coatings microcapsules were obtained. The resulting composition comprises telithromycin agglomerates 67.5%, ethylcellulose 7.5%, Eudragit® E 16.7%, talc 8.3%. The coated samples were easily dispersible in aqueous medium without any agglomeration or segregation phenomena.

### EXAMPLE 3

After carrying out a first step according to the Example 1 with 3 kg cyclohexane and a coating level of ethylcellulose of 9.5%, the second coating was performed over 92 mn, with using a Glatt GPCG1 fluid bed equipped with Wurster insert (4″), bottom plate <<A>> type, filter <<T165P>> type, 1 mm spraying nozzle and adding to 456 g of the first coated microcapsules, Eudragit® E100 10%, micronized talc 2.5%, ethanol 52.5%, purified water 35%. In order to avoid any damage to the ethylcellulose coating, the first part of the coating layer was applied at a lower spraying rate: 2.0 to 3.0 g/mn. Then at a spraying rate of 4.8 to 5.8 g/mn. The temperature of the air was 55 to 60° C. and the air speed 1.0 to 1.8 m/s. The atomisation pressure was 1.8 bars. The temperature of the product during the first part of the coating was between 33 and 38° C. The temperature of the product during the second part of the coating was between 30 and 32° C. The coating is performed with an amount of membrane of 20.9%. After drying on the fluid bed and sieving the corresponding 2 coatings microcapsules were obtained. The resulting composition comprises telithromycin agglomerates 71.6%, ethylcellulose 7.5%, Eudragit® E 16.7%, talc 4.2%. The coated samples were easily dispersible in aqueous medium nomena. The release is 97±1 after 2 mn.

We claim:

1. A composition comprising:
spherical agglomerates of telithromycin;
ethylcellulose;
at least one acrylic polymer; and
at least one anti-agglomeration agent
wherein the composition is in the form of microcapsules comprising two coating layers.

2. The composition of claim 1, wherein a first coating of said two coating layers is an ethylcellulose coating and a second coating of said two coating layers is an acrylic polymer coating.

3. The composition of claim 1, further comprising D(-) N-methylglucamine, and aluminium and magnesium silicate.

4. The composition of claim 3, further comprising at least one optional agent chosen from suspending agents, aromatization agents, sweetening agents and antimicrobial preservation agents.

5. The composition of claim 1, wherein the spherical agglomerates of telithromycin are present in an amount ranging from 40 to 65% by weight of the total composition.

6. The composition of claim 5, wherein the ethylcellulose is present in an amount ranging from 8 to 18% by weight of the total composition.

7. The composition of claim 6, wherein the acrylic polymer is present in an amount ranging from 18 to 35% by weight of the total composition.

8. The composition of claim 7, wherein the acrylic polymer is present in an amount ranging from 18 to 35% by weight of the total composition.

9. The composition of claim 8, further comprising
talc ranging from 4.5 to 10% by weight of the total composition
aluminium and magnesium silicate present in an amount ranging from 4 to 9% by weight of the total composition.

10. The composition of claim 8, further comprising
at least one additional component chosen from
xanthan gum present in an amount ranging from 0.3 to 0.7%
maltitol present in an amount ranging from 40 to 90%
sodium saccharinate present in an amount ranging from 0.6 to 1.4%; and
flavouring agent present in an amount ranging from 0.6 to 1.4%.

11. A process for preparing a composition of telithromycin, comprising
forming telithromycin into spherical agglomerates,
microencapsulating the spherical agglomerates with ethylcellulose, and fluid bed coating the microcapsules with at least one acrylic polymer.

12. The process of claim 11, wherein microcapsules are made by coacervation in cyclohexane.

13. The process of claim 11, further comprising adding D(-) N-methylglucamine and aluminium and magnesium silicate.

14. The process of claim 13, further comprising
adding at least one component chosen from suspending agents, aromatization agents, sweetening agents and antimicrobial preservation agents.

15. The process of claim 11, further comprising adding D(-) N-methylglucamine, aluminium and magnesium silicate, xanthane gum, maltitol, sodium saccharinate and flavouring agent.

16. A method of treating bacterial based infections comprising administering to a patient in need thereof an effective amount of the composition of claim 1.

17. A method for oral administration of telithromycin comprising orally administering a composition of claim 1 to a patient.

18. A method for taste masking telithromycin comprising
combining in the form of microcapsules comprising two coating layers
spherical agglomerates of telithromycin
ethylcellulose,
at least one acrylic polymer, and
at least one anti-agglomeration agent.
